(19) United States
(12) Patent Application Publication
(10) Pub. No.: US 2012/0202838 A1
(43) Pub. Date: Aug. 9, 2012

(54) DRUG FORMULATIONS

(75) Inventors: Soumojeet Ghosh, Gurnee, IL (US); Didier Lefebvre, Mundelein, IL (US); Bryan Wiesner, Lake Forest, IL (US); Joerg Breitenbach, Mannheim (DE)

(73) Assignees: Abbott Laboratories, Abbott Park, IL (US); Abbott GmbH & Co. KG, Wiesbaden (DE)

(21) Appl. No.: 13/289,982

(22) Filed: Nov. 4, 2011

Related U.S. Application Data
(60) Provisional application No. 61/410,339, filed on Nov. 4, 2010.

(51) Int. Cl. A61K 31/485; A61P 25/04

(52) U.S. Cl. 514/282

(57) ABSTRACT

In one embodiment, the present invention relates to abuse-deterrent drug formulations comprise a plurality of discrete domains uniformly dispersed in a pharmaceutically acceptable matrix, wherein said domains have high fracture toughness and comprise at least one polymer and at least one abuse-relevant drug. In another embodiment, the present invention relates to a formulation comprising a plurality of discrete mechanically reinforcing particles uniformly dispersed in a pharmaceutically acceptable matrix, wherein said matrix has high fracture toughness and comprises at least one polymer and at least one active agent, at least one abuse-relevant drug or a combination of at least one active agent and at least one abuse-relevant drug.

### RELATED APPLICATION INFORMATION

This application claims priority to U.S. application Ser. No. 61/410,339, filed on Nov. 4, 2010, the contents of which are herein incorporated by reference in their entirety.

### FIELD OF THE INVENTION

The present invention relates to abuse-deterrent and other tamper-resistant drug formulations. The invention further relates to methods for preparing such formulations.

### BACKGROUND OF THE INVENTION

Abuse of drugs contained in medicaments is a major problem. In particular, medicaments containing opioid drugs are prone to abuse. Such abuse may manifest itself in attempts to separate the opioids or other desired components from the medicaments for intended abuse.

With regard to abuse by injection, the opioid containing pharmaceutical form is reduced to a fine powder using household means like a coffee grinder, or even simply by chewing or biting the pharmaceutical form. The rough powder obtained can then be extracted in a small volume of liquid such as an alcoholic beverage. The liquid obtained can then be roughly filtered, e.g., using a cigarette filter, before it is injected via intravenous route. In this case, the active agent then becomes immediately available in the bloodstream, giving rise to an immediate psychotropic effect sought by drug addicts.

Abuse by inhalation also consists of crushing the tablet until a sufficiently fine powder is obtained to render the active agent accessible to the micro-vessels of the intranasal mucous membrane.

There have been attempts to impart abuse-deterrent features to drug formulations. For example, WO 2007/085024 discloses such formulations wherein the extractability of the abuse-relevant drug is confined to certain limits WO 2005/079760 relates to tamper-resistant controlled-release formulations, which are based on a rubbery matrix including a neutral poly(ethyacrylate, methyl methacrylate) copolymer and an (abuse-relevant) active agent.

Despite previous efforts there is a continuing need for tamper-resistant drug formulations.

U.S. Pat. No. 6,488,963 B1 discloses the utility of poly(ethylene oxide) for controlled-release formulations of a wide range of pharmaceutically active agents but does not address the issue of abuse-deterrent drug formulations.

There is also a need to prevent the tampering of formulations containing active ingredients. In particular, in certain countries, pharmacists are authorized to modify commercially available dosage forms and reformulate and reshape such dosage forms. Depending on the active agent, such reformulation and reshaping can be highly problematic.

### SUMMARY

Surprisingly, it has now been found that formulations of the present invention comprising domains having high fracture toughness, which are dispersed in pharmaceutically acceptable matrices and contain active agents and/or abuse-relevant drugs, are suitable as unique drug formulations. Furthermore, it has now been found that certain formulations offer high fracture resistance having unique mechanical properties, as the ability to be plastically reshaped without heating.

In one embodiment, the present invention relates to an abuse-deterrent drug formulation comprising a plurality of discrete domains uniformly dispersed in a pharmaceutically acceptable matrix, wherein said domains have high fracture toughness and comprise at least one polymer and at least one abuse-relevant drug.

Typically, high fracture toughness is imparted by a high loss modulus polymer. The high loss modulus polymer can have a tan delta maximum at a temperature of about 50° C. or below, e.g., maximum mechanical energy dissipation (a tan delta maximum) at a temperature in the range of from about 25° C. to about 50° C. High fracture toughness may be imparted by semi-crystallinity or by a high loss modulus in combination with semi-crystallinity.

Further aspects comprise drug formulations, wherein the matrix, or both the matrix and the domains comprise at least one further active agent, and/or formulations, wherein the domains comprise at least one further abuse-deterrent drug.

Further aspects comprise drug formulations, wherein the domains have an average size of about 100 micrometer to about 1000 micrometer.

According to further aspects, the polymer contained in the domains is a poly(ethylene oxide) having a molecular weight of about 100,000 to about 10,000,000 Daltons.

In still yet further aspects, the domains additionally comprise a plasticizer. In particular, said plasticizer is a poly(ethylene oxide) having a molecular weight less than about 900,000 Daltons. Alternatively, the plasticizer is a poloxamer.

In still further aspects, the weight ratio of said abuse-relevant drug and the polymer contained in the domains is form about 50:50 to about 0.1 to 99.9.

According to further aspects, the matrix comprises at least one pharmaceutically acceptable polymer selected form cellulose ethers, cellulose ethers, and (meth)acrylic polymers, specifically, hydroxypropyl methylcellulose and/or an ionic, more specifically, a cationic (meth)acrylic polymer.

In particular aspects, the (meth)acrylic polymer is a ionic (meth)acrylic polymer comprising quaternary ammonium groups.

In further aspects, the abuse-relevant drug is selected from analgesics, sedatives, anxyolytics, psychostimulants, anesthetics, antidepressants, antipsychotics, or combinations thereof. In particular embodiments, the abuse-relevant drug is selected from benzodiazepines, amphetamines, propofol, midazolam, tricyclic antidepressants, monoamine oxidase inhibitors, clozapine, amisulpride, olanzapine, and risperidone.

In particular aspects, the abuse-relevant drug is an analgesic an opoid, in particular an opioid selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, levorphanol, lofentanil, meperidine, meptazinol, metazocine, methadone, metopon, morphine, myrophine, nalbulphine, narceine, nicomorphine, norpipanone, opium, oxycodone, oxymorphone, papvretum, paladone, pentazocine, phenadoxone, phenazocine, phenomorphan, phenoperidine, piminodine, propiram, propoxyphene, sufentanil, tapenadol, tilidine, and tramadol, and salts, esters, prodrugs and mixtures thereof. In particular, the opoid is hydrocodone.

According to particular aspects, the further active agent is selected from the group consisting of salicylates, anthranilic acid derivatives, arylacetic acid derivatives, arylpropionic acid derivatives, oxicames and pyrazolideindiones, and in particular can be paracetamole.

In another embodiment, the invention further relates to method of preparing an abuse-deterrent drug formulation. The method comprises the steps of:
a) providing particles which comprise a fracture toughness-imparting polymer and at least one abuse-relevant drug, and
b) embedding a plurality of said particles in a pharmaceutically acceptable matrix in a unit dosage form.

According to one aspect, said particles are prepared by hot-melt extruding a formable composition comprising the polymer and the abuse-relevant drug; and sizing the extruded solid to form particles. In particular, the particles may be hot-spheronized to form spheronized particles.

In one further aspect the particles are prepared by one of spray drying, or wet granulating a composition comprising the polymer and the abuse-relevant drug.

In one particular aspect, step b) of the method comprises blending said particles with a compressible powder to yield a powder blend and compressing the powder blend into a unit dosage form. In an alternative aspect, step b) comprises liquefying, melting or softening a matrix composition by heating, dispersing said particles in the liquefied (semi solid) matrix composition, shaping the liquefied (semi solid) composition into a unit dosage form, and solidifying the composition.

In another embodiment, the present invention relates to a formulation comprising a plurality of discrete mechanically reinforcing particles uniformly dispersed in a pharmaceutically acceptable matrix, wherein said matrix has high fracture toughness and comprises at least one polymer and at least one active agent, at least one abuse-relevant drug or a combination of at least one active agent and at least one abuse-relevant drug.

In the above method, the polymer has a maximum mechanical energy dissipation (tan delta maximum) at a temperature of about 50° C. or below.

In the above method, the polymer is semi-crystalline.

In the above method, the polymer is a semi-crystalline polymer having a maximum mechanical energy dissipation (tan delta maximum) at a temperature of about 50° C. or below.

In the above method, the mechanically reinforcing particles are composed of a filler, a fiber, or of a combination of a filler and a fiber. The filler that can be used is dicalcium phosphate. The fiber that can be used can comprise a cellulosic excipient or any other pharmaceutically acceptable fibrous material.

In the above method, the polymer comprises at least one poly(ethylene oxide). Specifically, the poly(ethylene oxide) has a molecular weight of about 100,000 to about 10,000,000 Daltons.

In the above method, the matrix additionally comprises a plasticizer. Specifically, the plasticizer is a poly(ethylene oxide) having a molecular weight less than about 900,000 Daltons.

In the above method, the matrix additionally comprises an anti-oxidant to protective the active agent or abuse-relevant drug.

In the above method, the weight ratio of active agent, abuse-relevant drug or combination of active agent and abuse-relevant drug and the polymer is from about 50:50 to about 0.1 to 99.9.

In the above method, the abuse-relevant drug is an opiod. The opiod can be selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, levorphanol, lofentanil, meperidine, meptazinol, metazocine, methadone, metopon, morphine, myrophine, nalbulphine, narceine, nicomorphine, norpipanone, opium, oxycodone, oxymorphone, papvretum, paladone, pentazocine, phenadoxone, phenazocine, phenomorphan, phenoperidine, piminodine, propiram, propoxyphene, sufentanil, tapenadol, tilidine, and tramadol, and salts, esters, prodrugs and mixtures thereof. More specifically, the opiod is hydrocodone.

In another embodiment, the present invention relates to a method of preparing a formulation, the method comprising the steps of:
a) manufacturing particles which comprise a fracture toughness-imparting polymer and at least one active agent, at least one abuse-relevant drug or a combination of at least one active agent and at least one abuse-relevant drug, and
b) embedding a plurality of said particles in a pharmaceutically acceptable matrix in a unit dosage form.

In the above method, said particles comprise a filler or a fiber. For example, the filler is dicalcium phosphate.

### DETAILED DESCRIPTION OF THE INVENTION

### I. Definitions

Section headings as used in this section and the entire disclosure herein are not intended to be limiting.

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

As used herein, the term “about” is used synonymously with the term “approximately.” Illustratively, the use of the term “about” indicates that values slightly outside the cited values, namely, plus or minus 10%. Such dosages are thus encompassed by the scope of the claims reciting the terms “about” and “approximately.”

As used herein, the phrase “abuse-relevant” drug refers to any biologically effective substance or composition, which is subject to regulatory restrictions. Abuse-relevant drugs also refer to substances or compositions, which are contained in pharmaceuticals and which are prone to attempts of being extracted or separated from the pharmaceutical composition. Said extracted or enriched substance might later be abused for purposes not intended by the original pharmaceutical. For example, opiates comprised in pharmaceuticals for synergistic effects together with pain-killing substances, might be extracted to satisfy the needs of opiate addicts. “Abuse-deterrent” drug formulations are characterized by features which render a separation or extraction of the abuse-relevante drug from its pharmaceutical formulation, e.g. by mechanical, physical or chemical means, more difficult or impossible. Examples of abuse-relevant drugs include:

Analgesics, such as, for example; Opioids, Natural opium alkaloids, semi-synthetic opium alkaloids, Morphine, Opium, Hydromorphone, Nicomorphine, Oxycodone, Dihydrocodeine, Diamorphine, Papaveretum, Codeine, Phenylpiperidine derivatives, Ketobemidone, Pethidine, Fentanyl, Diphenylpropylamine derivatives, Dextromoramide, Piritramide, Dextropropoxyphene, Bezitramide, Methadone, Benzomorphan derivatives, Pentazocine, Phenazocine, Oripavine derivatives, Buprenorphine, Morphinan derivatives, Butorphanol, Nalbuphine, Tilidine, Tramadol, Dezocine, Salicylic acid and derivatives, Acetylsalicylic acid, Aloxiprin, Choline salicylate, Sodium salicylate, Salicylamide, Salsalate, Ethenzamide, Morpholine salicylate, Dipyrocetyl, Benorilate, Diflunisal, Potassium salicylate, Guacetisal, Carbasalate calcium, Imidazole salicylate, Pyrazolones, Phenazone, Metamizole sodium, Aminophenazone, Propyphenazone, Nifenazone, Anilides, Paracetamol, Phenacetin, Bucetin, Propacetamol, Other analgesics and antipyretics, Rimazolium, Glafenine, Floctafenine, Viminol, Nefopam, Flupirtine, Ziconotide, Allylprodine, Prodine, Alphaprodine, Betaprodine, Anileridine, Benzylmorphine, Bezitramide, Buprenorphine, Clonitazene, Diampromide, Dihydromorphine, Dimenoxadol, Dimepheptanol, Dimethylthiambutene, Dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, levorphanol, lofentanil, meperidine, meptazinol, metazocine, methadone, metopon, morphine, myrophine, nalbulphine, narceine, nicomorphine, norpipanone, opium, oxycodone, oxymorphone, papvretum, paladone, pentazocine, phenadoxone, phenazocine, phenomorphan, phenoperidine, piminodine, propiram, propoxyphene, sufentanil, tapenadol, tilidine, and tramadol;
Anesthetics, such as, for example; Ethers, Diethyl ether, Vinyl ether, Halogenated hydrocarbons, Halothane, Chloroform, Methoxyflurane, Enflurane, Trichloroethylene, Isoflurane, Desflurane, Sevoflurane, Barbiturates, Methohexital, Hexobarbital, Thiopental, Narcobarbital, Opioid anesthetics, Fentanyl, Alfentanil, Sufentanil, Phenoperidine, Anileridine, Remifentanil, Other general anesthetics, Droperidol, Ketamine, Propanidid, Alfaxalone, Etomidate, Propofol, Hydroxybutyric acid, Nitrous oxide, Esketamine, Xenon, Esters of aminobenzoic acid, Metabutethamine, Procaine, Tetracaine, Chloroprocaine, Benzocaine, Amides, Bupivacaine, Lidocaine, Mepivacaine, Prilocaine, Butanilicaine, Cinchocaine, Etidocaine, Articaine, Ropivacaine, Levobupivacaine, Esters of benzoic acid, Cocaine, Other local anesthetics, Ethyl chloride, Dyclonine, Phenol, Capsaicin;
Antiepileptic drug substances such as, for example; Barbiturates and derivatives, Methylphenobarbital, Phenobarbital, Primidone, Barbexaclone, Metharbital, Hydantoin derivatives, Ethotoin, Phenytoin, Amino(diphenylhydantoin) valeric acid, Mephenytoin, Fosphenytoin, Oxazolidine derivatives, Paramethadione, Trimethadione, Ethadione, Succinimide derivatives, Ethosuximide, Phensuximide, Mesuximide, Benzodiazepine derivatives, Clonazepam, Carboxamide derivatives, Carbamazepine, Oxcarbazepine, Rufinamide, Fatty acid derivatives, Valproic acid, Valpromide, Aminobutyric acid, Vigabatrin, Progabide, Tiagabine, Other antiepileptics, Sultiame, Phenacemide, Lamotrigine, Felbamate, Topiramate, Gabapentin, Pheneturide, Levetiracetam, Zonisamide, Pregabalin, Stiripentol, Lacosamide, Beclamide;
Antipsychotic drug substances, such as, for example; Phenothiazines with an aliphatic side-chain, Chlorpromazine, Levomepromazine, Promazine, Acepromazine, Triflupromazine, Cyamemazine, Chlorproethazine, Phenothiazines with piperazine structure, Dixyrazine, Fluphenazine, Perphenazine, Prochlorperazine, Thiopropazate, Trifluoperazine, Acetophenazine, Thioproperazine, Butaperazine, Perazine, Phenothiazines with piperidine structure, Periciazine, Thioridazine, Mesoridazine, Pipotiazine, Butyrophenone derivatives, Haloperidol, Trifluperidol, Melperone, Moperone, Pipamperone, Bromperidol, Benperidol, Droperidol, Fluanisone, Indole derivatives, Oxypertine, Molindone, Sertindole, Ziprasidone, Thioxanthene derivatives, Flupentixol, Clopenthixol, Chlorprothixene, Tiotixene, Zuclopenthixol, Diphenylbutylpiperidine derivatives, Fluspirilene, Pimozide, Penfluridol, Diazepines, oxazepines and thiazepines, Loxapine, Clozapine, Olanzapine, Quetiapine, Neuroleptics, in tardive dyskinesia, Tetrabenazine, Benzamides, Sulpiride, Sultopride, Tiapride, Remoxipride, Amisulpride, Veralipride, Levosulpiride, Lithium, Other antipsychotics, Prothipendyl, Risperidone, Clotiapine, Mosapramine, Zotepine, Aripiprazole, Paliperidone;
Hypnotic and sedative drug substances, such as, for example; Barbiturates, Pentobarbital, Amobarbital, Butobarbital, Barbital, Aprobarbital, Secobarbital, Talbutal, Vinylbital, Vinbarbital, Cyclobarbital, Heptabarbital, Reposal, Methohexital, Hexobarbital, Thiopental, Etallobarbital, Allobarbital, Proxibarbal, Aldehydes and derivatives, Chloral hydrate, Chloralodol, Acetylglycinamide chloral hydrate, Dichloralphenazone, Paraldehyde, Benzodiazepineemepronium derivatives, Flurazepam, Nitrazepam, Flunitrazepam, Estazolam, Triazolam, Lormetazepam, Temazepam, Midazolam, Brotizolam, Quazepam, Loprazolam, Doxefazepam, Cinolazepam, Piperidinedione derivatives, Glutethimide, Methyprylon, Pyrithyldione, Benzodiazepine related drugs, Zopiclone, Zolpidem, Zaleplon, Ramelteon, Other hypnotics and sedatives, Methaqualone, Clomethiazole, Bromisoval, Carbromal, Scopolamine, Propiomazine, Triclofos, Ethchlorvynol, Valerian, Hexapropymate, Bromides, Apronal, Valnoctamide, Methylpentynol, Niaprazine, Melatonin, Dexmedetomidine, Dipiperonylaminoethanol;
Anxiolytic drug substances, such as, for example; Benzodiazepine derivatives, Diazepam, Chlordiazepoxide, Medazepam, Oxazepam, Potassium clorazepate, Lorazepam, Adinazolam, Bromazepam, Clobazam, Ketazolam, Prazepam, Alprazolam, Halazepam, Pinazepam, Camazepam, Nordazepam, Fludiazepam, Ethyl loflazepate, Etizolam, Clotiazepam, Cloxazolam, Tofisopam, Diphenylmethane derivatives, Hydroxyzine, Captodiame, Carbamates, Meprobamate, Emylcamate, Mebutamate, Dibenzo-bicyclo-octadiene derivatives, Benzoctamine, Azaspirodecanedione derivatives, Buspirone, Other anxiolytics, Mephenoxalone, Gedocamil, Etifoxine. Antidepressant drug substances, such as, for example tricyclic antidepressants, non-selective monoamine reuptake inhibitors, Desipramine, Imipramine, lmipramine oxide, Clomipramine, Opipramol, Trimipramine, Lofepramine, Dibenzepin, Amitriptyline, Nortriptyline, Protriptyline, Doxepin, Iprindole, Melitracen, Butriptyline, Dosulepin, Amoxapine, Dimetacrine, Amineptine, Maprotiline, Quinupramine, Selective serotonin reuptake inhibitors, Zimeldine, Fluoxetine, Citalopram, Paroxetine, Sertraline, Alaproclate, Fluvoxamine, Etoperidone, Escitalopram, Monoamine oxidase inhibitors, non-selective, Isocarboxazid, Nialamide, Phenelzine, Tranylcypromine, Iproniazide, Iproclozide, Monoamine oxidase A inhibitors, Moclobemide, Toloxatone, Other antidepressants, Oxitriptan, Tryptophan, Mianserin, Nomifensine, Trazodone, Nefazodone, Minaprine, Bifemelane, Viloxazine, Oxaflozane, Mirtazapine, Medifoxamine, Tianeptine, Pivagabine, Venlafaxine, Milnacipran, Reboxetine, Gepirone, Duloxetine, Agomelatine, Desvenlafaxine, Centrally acting sympathomimetics, Amphetamine, Dexamphetamine, Metamphetamine, Methylphenidate, Pemoline, Fencamfamin, Modafinil, Fenozolone, Atomoxetine, Fenetylline, Xanthine derivatives, Caffeine, Propentofylline, Other psychostimulants and nootropics, Meclofenoxate, Pyritinol, Piracetam, Deanol, Fipexide, Citicoline, Oxiracetam, Pirisudanol, Linopirdine, Nizofenone, Aniracetam, Acetylcarnitine, Idebenone, Prolintane, Pipradrol, Pramiracetam, Adrafinil, Vinpocetine; Drug substances used in addictive disorders, such as, for example; Nicotine, Bupropion, Varenicline, Disulfiram, Calcium carbimide, Acamprosate, Naltrexone, Buprenorphine, Methadone, Levacetylmethadol, Lofexidine. Antivertigo drug substances, such as, for example; Betahistine, Cinnarizine, Flunarizine, Acetylleucine, other nervous system drugs, Gangliosides and ganglioside derivatives, Tirilazad, Riluzole, Xaliproden, Hydroxybutyric acid, Amifampridine;
Further abuse-relevant drugs are Ethylmorphine, Codeine, Opium alkaloids with morphine, Normethadone, Noscapine, Pholcodine,
Dextromethorphan, Thebacon, Dimemorfan, Acetyldihydrocodeine, Benzonatate, Benproperine, Clobutinol, Isoaminile, Pentoxyverine, Oxolamine, Oxeladin, Clofedanol, Pipazetate, Bibenzonium bromide, Butamirate, Fedrilate, Zipeprol, Dibunate, Droxypropine, Prenoxdiazine, Dropropizine, Cloperastine, Meprotixol, Piperidione, Tipepidine, Morclofone, Nepinalone, Levodropropizine, Dimethoxanate; Further abuse-relevant drugs are opioid agonists/antagonists such as Cyclazonine opiate analogues such as Desomorphine.

In one aspect, the abuse-deterrent drug is an opoid. More specifically, the opiod is hydrocodone or a pharmaceutically acceptable salt or hydrate thereof. In another aspect, the hydrocodone is hydrocodone bitartrate hemipentahydrate (namely, hydrocodone bitartrate according to USP/NF).

The phrase “active agent” as used herein refers to one or more chemical entities (or pharmaceutically acceptable salts thereof) that display certain pharmacological effects in a subject and are administered for such purpose. The term “active agent”, “active agent” and “drug” are used interchangeably herein. The form of the active agent used in preparing the dosage forms of the present invention is not critical. For example, active agent used in the method of the present invention can be amorphous or crystalline. The crystalline nature of the active agent can be detected using powder X-ray diffraction analysis, by differential scanning calorimetry or any other techniques known in the art. Examples of active agents that can be used in the present invention are:

Antiinflammatory and antirheumatic drug substances, such as, for example; Butylpyrazolidines, Phenylbutazone, Mofebutazone, Oxyphenbutazone, Clofezone, Kebuzone, Acetic acid derivatives and related substances, Indometacin, Sulindac, Tolmetin, Zomepirac, Diclofenac, Alclofenac, Bumadizone, Etodolac, Lonazolac, Fentiazac, Acemetacin, Difenpiramide, Oxametacin, Proglumetacin, Ketorolac, Aceclofenac, Bufexamac, Oxicams, Piroxicam, Tenoxicam, Droxicam, Lornoxicam, Meloxicam, Propionic acid derivatives, Ibuprofen, Naproxen, Ketoprofen, Fenoprofen, Fenbufen, Benoxaprofen, Suprofen, Pirprofen, Flurbiprofen, Indoprofen, Tiaprofenic acid, Oxaprozin, Ibuproxam, Dexibuprofen, Flunoxaprofen, Alminoprofen, Dexketoprofen, Fenamates, Mefenamic acid, Tolfenamic acid, Flufenamic acid, Meclofenamic acid, Coxibs, Celecoxib, Rofecoxib, Valdecoxib, Parecoxib, Etoricoxib, Lumiracoxib, Nabumetone, Niflumic acid, Azapropazone, Glucosamine, Benzydamine, Glucosaminoglycan polysulphate, Proquazone, Orgotein, Nimesulide, Feprazone, Diacerein, Morniflumate, Tenidap, Oxaceprol, Chondroitin sulphate, Feprazone, Dipyrocetyl, Acetylsalicylic acid, Quinolines, Oxycinchophen, Gold preparations, Sodium aurothiomalate, Sodium aurotiosulphate, Auranofin, Aurothioglucose, Aurotioprol, Penicillamine, Bucillamine;

Antimigraine drug substances, such as, for example; Ergot alkaloids, Dihydroergotamine, Ergotamine, Methysergide, Lisuride, Corticosteroid derivatives, Flumedroxone, Selective serotonin (5HT1) agonists, Sumatriptan, Naratriptan, Zolmitriptan, Rizatriptan, Almotriptan, Eletriptan, Frovatriptan, Other antimigraine preparations, Pizotifen, Clonidine, Iprazochrome, Dimetotiazine, Oxetorone;

Anticholinergic drug substances, such as, for example; Tertiary amines, Trihexyphenidyl, Biperiden, Metixene, Procyclidine, Profenamine, Dexetimide, Phenglutarimide, Mazaticol, Bomaprine, Tropatepine, Ethers chemically close to antihistamines, Etanautine, Orphenadrine (chloride), Ethers of tropine or tropine derivatives, Benzatropine, Etybenzatropine;

Dopaminergic ative substances, such as, for example; Dopa and dopa derivatives, Levodopa, Melevodopa, Etilevodopa, Adamantane derivatives, Amantadine, Dopamine agonists, Bromocriptine, Pergolide, Dihydroergocryptine mesylate, Ropinirole, Pramipexole, Cabergoline, Apomorphine, Piribedil, Rotigotine, Monoamine, oxidase B inhibitors, Selegiline, Rasagiline, other dopaminergic agents, Tolcapone, Entacapone, Budipine;

Anti-dementia drug substances, such as, for example; Anticholinesterases, Tacrine, Donepezil, Rivastigmine, Galantamine, Other anti-dementia drugs, Memantine, Ginkgo biloba; and other nervous system drug substances, such as, for example; Parasympathomimetics, Anticholinesterases, Neostigmine, Pyridostigmine, Distigmine, Ambenonium, Choline esters, Carbachol, Bethanechol, Other parasympathomimetics, Pilocarpine, Choline alfoscerate.

Examples of other active agents include antibiotics, analgesics, vaccines, anti-diabetic agents, antifungal agents, antineoplastic agents, anti-parkinsonian agents, anti-viral agents (such as, for example, amprenavir (Agenerase), atazanavir (Reyataz), fosamprenavir (Lexiva), indinavir (Crixivan), lopinavir, ritonavir (norvir), nelfinavir (Viracept), saquinavir (Invirase), tipranavir (Aptivus), brecanavir, darunavir (Prezista)), appetite suppressants, biological response modifiers, cardiovascular agents, central nervous system stimulants, chemotherapeutic agents (such as, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a BcI-2 inhibitor, a Bcl-xl inhibitor, an HDAC inhibitor, a c-MET inhibitor, a PARP inhibitor (such as, for example, PJ34, AG14699, AG14361, CEP-6800, CEP-8983, INO-1001, KU59436, BSI-201, GPI 21016, GPI15427 or AZD2281), a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380, chlamydocin, JNJ-16241199, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]- benzoyl]-, disodium salt, heptahydrate, camptothecin, irinotecan; PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-0-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa), contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, respiratory agents, skin and mucous membrane agents, smoking cessation agents, steroids, urinary tract agents, uterine relaxants, vaginal agents, vasodilator, anti-hypertensive, hyperthyroids, anti-hyperthyroids, anti-asthmatics and vertigo agents. Furthermore, the active agent can further include salicylates, anthranilic acid derivatives, arylacetic acid derivatives, arylpropionic acid derivatives, oxicames and pyrazolideindiones (such as paracetamole).

The terms “administer”, “administering”, “administered” or “administration” refer to any manner of providing an abuse-relevant drug, active agent or a combination of an abuse-relevant drug and active agent (or a pharmaceutically acceptable salt thereof) to a subject or patient. Routes of administration can be accomplished through any means known by those skilled in the art. Such means include, but are not limited to, oral, buccal, intravenous, subcutaneous, intramuscular, transdermal, by inhalation and the like.

As used herein, the term “discrete domains” are defined as common in the technology of disperse systems and refer to particles, which are embedded in the pharmaceutically acceptable matrix and can be discerned from said matrix by phase boundaries. The discrete domains are “uniformely dispersed” in the pharmaceutically acceptable matrix, if there are essentially no local accumulation or depletion of discrete domain in any region of the matrix.

As used herein, the phrase “low molecular weight PEO” is intended to mean poly(ethylene oxide) homopolymer having an average molecular weight less than about 900,000.

By “pharmaceutically acceptable,” refers in a broad sense to compounds, materials, compositions and/or dosage forms, which are, within the scope of sound medical judgment, suitable for use in contact with tissues of a subject without excessive toxicity, irritation, allergic response, or other problems or complications, commensurate with a reasonable benefit/risk ratio.

As used herein, the phrase “high loss modulus polymer” encompasses polymeric materials that have elevated loss modulus at impact frequencies corresponding to commonly used methods of grinding and crushing. The elevated loss modulus ensures that a high portion of impact energy is dissipated into heat rather than in the formation cracks leading to fracture. Loss modulus can be expressed as loss factor tan delta (tan δ) in the dynamic viscoelasticity measurement. Dynamic viscoelasticity measurement is described, for example, in “Viscoelastic properties of polymers”, John Ferry, John Wiley & Sons, ISBN 0-471-04894-1, 1980. The tan delta maximum occurs at a temperature and frequency regime corresponding to maximum viscous loss. The temperature and frequency at which tan delta reaches a maximum may be derived experimentally using a technique called Dynamic Mechanical Analysis (DMA). Suitable polymers have a tan delta maximum at a temperature of about 50° C. or below, e.g., in the range of from about 25° C. to about 50° C. at frequencies ranging from 1 Hz to 25 kHz, for example at 10 Hz.

In one aspect, the high loss modulus polymers are also semi-crystalline. The high loss modulus polymer can be a hydrophilic polymer that is capable of swelling or gelling in an aqueous or other solvent environment. The high loss modulus polymer is pharmaceutically acceptable.

Suitable examples of the polymer materials for forming crush-resistant particles are poly(ethylene oxides) or “PEO”, in particular those with a molecular weight of about 100,000 to 10,000,000 Daltons, for example 1,000,000 to about 10,000,000 Daltons. “Molecular weight” means the weight average molecular weight of an essentially monomodal molecular weight distribution. Mixtures of different PEO grades can advantageously be used. In these instances, the molecular weight distribution will be bimodal (or multimodal). Relative amounts and molecular weights of the individual PEO grades can be selected such that the weight-averaged molecular weight of the PEO composition is within the limits given before.

The polyethylene oxide used in a directly compressible formulation of the present invention can be a homopolymer having repeating oxyethylene groups, i.e.,—(—O—CH₂—CH₂—)ₙ—, where n can range from about 2,000 to about 180,000. The polyethylene oxide is a commercially available and pharmaceutically acceptable homopolymer. These PEO polymers fulfill the specifications as outlined in the US Pharmacoepia. Examples of suitable, commercially available polyethylene oxide polymers include Polyox®, Sumitomo grader, WSRN-105 and/or WSR-coagulant, available from Dow chemicals. In another aspect, the polymer can be a copolymer, such as a block copolymer of PEO and PPO.

Other suitable examples of the polymer for forming crush-resistant particles include carbomers. In one aspect, the carbomers can have a molecular weight ranging from 700,000 to about 4,000,000,000. In one embodiment, the viscosity of the polymer can range from about 4000 to about 39,400 cps. Examples of suitable, commercially available carbomers include polyacrylic acids such as carbopol 934P NF, carbopol 974P NF and carbopol 971P NF, available from Noveon Pharmaceuticals.

As used herein, the phrase, “discrete mechanically reinforcing domains” refers to particles, which are embedded in the pharmaceutically acceptable matrix that provide or impart mechanical strength to the matrix and can be discerned from said matrix by phase boundaries. The discrete domains are “uniformely dispersed” in the pharmaceutically acceptable matrix, if there are essentially no local accumulation or depletion of discrete domain in any region of the matrix.

As used herein, the term “plasticizer” refers to all compounds capable of plasticizing the polymer contained in the domains, in particular high molecular weight PEO. The plasticizer should be able to lower the glass transition temperature or softening point of the PEO, in order to allow for lower processing temperature, extruder torque and pressure during the hot-melt extrusion process. Plasticizers, such as PEG and low molecular weight PEO, generally broaden the average molecular weight of the PEO thereby lowering its glass transition temperature or softening point. Plasticizers also generally reduce the viscosity of a polymer melt thereby allowing for lower processing temperature and extruder torque during hot-melt extrusion.

The plasticizer employed in the present invention may be a solvent for the PEO.

Plasticizers useful in the invention include, by way of example and without limitation, low molecular weight polymners, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene oxide) (average molecular weight less than about 900,000) and poly(ethylene glycol).

Such plasticizers may be ethylene glycol, propylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, triethyl citrate, acetyl triethyl citrate, tributyl citrate and allyl glycolate. All such plasticizers are commercially available from sources such as Aldrich or Sigma Chemical Co. In one aspect, the plasticizer that can be employed is a poloxamer, such as poloxamer 407 (Lutrol F127). The plasticizer can be a solid at room temperature. In this case the plasticizer in powder form can be homogenously blended with the other components of the formulation prior to processing (e.g. extrusion). A plasticizer which is liquid at room temperature can be pumped into the extruder directly. Plasticizers which are solid at room temperature but having a low melting point can be pumped at higher temperature in liquid form into the extruder.

The PEG based plasticizers are available commercially or may be made by a variety of methods, such as disclosed in Poly (ethylene glycol) Chemistry: Biotechnical and Biomedical Applications (J. M. Harris, Ed.; Plenum Press, N.Y.) the teachings of which are hereby incorporated by reference.

The amount of plasticizer used in the formulation will depend upon its composition, physical properties, effect upon the polymer to be plasticized, interaction with other components of the formulation, ability to solubilize the therapeutic compound or other factors to be considered in the preparation of pharmaceutical formulations. The amount of plasticizer present in the formulation affects its properties. By way of example, when the plasticizer is PEG, its content will generally not exceed about 40% weight of the formulation.

When present, the relative amount of plasticizer used may be expressed by the weight ratio of polymer: plasticizer, and will generally fall in the range of about 100:0 to about 60:40, about 100:0 to about 85:15 and about 100:0 to about 90:10. The amount of plasticizer will generally not exceed the amount of the polymer.

Likewise, suitable examples for plasticizers are poly(ethylene oxides) having a lower molecular weight compared to the high-molecular weight poly(ethylene oxides), for example a molecular weight corresponding to 10% to 75% of the molecular weight of the respective high-molecular weight poly(ethylene oxide), such as a molecular weight lower by 75%, 60%, 50%, 45%, 40%, 35%, 30%, 25%, 20%, 15% or 10%. In particular, the plasticizer may be poly(ethylene oxide) having a molecular weight less than about 500,000 Daltons. Further examples of suitable low molecular weight poly(ethylene oxides) are those with a viscosity ranging from 40 to 55 mPa.s or from 55 to 75 mPa.s (determined as 5% solution) and having a solution pH ranging from 6 to 8. Examples of suitable high molecular weight poly(ethylene oxides) are those with a viscosity ranging from 1650 to 5500 mPa.s (1% solution), from 5500 to 7500 mPa.s (1% solution), and from 5500 to 7500 mPa.s (1% solution), each of which having a solution pH ranging from 6 to 8.

As used herein, “semi-crystalline” refers to polymers that exist as viscous liquids at temperatures above the melting point of the crystals. Upon cooling, crystals nucleate and grow to locally. These polymers are referred to as “semicrystalline” become some fraction of the polymer remains un-crystallized, or, amorphous when the polymer is cooled to room temperature. The amorphous polymer becomes trapped between the growing crystals. As a result of the highly entangled nature of the polymer chains, the movement of the amorphous polymer becomes restricted.

The term “subject” refers to an animal. In one aspect, the animal is a mammal, including a human or non-human. The terms patient and subject may be used interchangeably herein.

The phrase “substantially crystalline” when used in connection with the formulations of the present invention means that the formulation includes an active agent, an abuse-relevant drug or a combination of an active agent and an abuse-relevant drug that is present in the formulation in an amount greater than 70% crystalline, 75% crystalline, 80% crystalline, 85% crystalline, 90% crystalline, 95% crystalline, 96% crystalline, 97% crystalline, 98% crystalline, 99% crystalline or 100% crystalline as determined by as determined by X-ray diffraction and/or polarized light microscopy (PLM) as known in the art. See, e.g., Remington: The Science and Practice of Pharmacy, 21.sup.st edition, Lippincott, Williams and Wilkins, Baltimore, Md. (2005); Byrn et al., Solid State Chemistry of Drugs, supra; The United States Pharmacopeia, (1995) 23rd ed. In certain embodiments, a crystal form of a substance may be substantially free of one or more amorphous forms and/or other crystal forms.

### II. Abuse-Deterrent Drug Formulations

In one embodiment, the present invention relates to an abuse-deterrent drug formulation comprising a plurality of discrete domains uniformly dispersed in a pharmaceutically acceptable matrix, wherein said domains have high fracture toughness and comprise at least one polymer and at least one abuse-relevant drug. In one aspect, the at least one polymer is a semi-crystalline polymer, a high loss modulus polymer or a combination of a semi-crystalline polymer and a high loss modulus polymer. When the pharmaceutically acceptable matrix contains PEO, the formulations of the present invention are shapeable at room temperature unlike other formulations known in the art which have to re-heated to be re-shaped Impurities are introduced into such formulations when they are re-heated for the purposes of re-shaping.

The “plurality” of domains as used in reference to the formulations of the present invention refers to at least two domains. In one aspect, the abuse-deterrent drug formulation is in unit-dosage form, thus containing, within acceptable limits of variation, a predetermined amount of one or more abuse-relevant drugs and optionally of at least one further active agent. It is clear that the number of domains depends both on the absolute amount of those drug(s) to be incorporated in the abuse-deterrent drug formulation and on the size of the domains carrying those drug(s). The absolute amount of drug to be incorporated in turn depends on the nature of the drug itself and the target group of patients to be treated with the drug, and can routinely be determined by those skilled in the art.

The semi-crystalline polymer, high loss modulus polymer or combination of semi-crystalline polymer and high loss modulus polymer provide abuse-deterrent features to the drug formulation by confining the abuse-relevant drug to discrete domains within the drug formulation. Those domains, even after separation from their surrounding matrix, provide protection against abuse.

Crushing of a pharmaceutical form is a commonly used step for misuse of said pharmaceutical formulation. The polymeric domains of the inventive drug formulation resist crushing since they possess rubbery or resilient properties, thereby rendering attempts to chemically extract the abuse-relevant drug after crushing or to inhale powders of the crushed particles more difficult.

The domains have dimensions large enough to provide deterrence against sniffing and injection but not so large as to fail content uniformity requirements for the abuse-relevant drug. The size of the domains affects content uniformity, because the larger the domains, the higher is the impact of fluctuations in the number of domains incorporated into a single dosage form. The drug formulations of the present invention meet official content uniformity requirements, in particular those specified by the US Pharmacopeia which allow a maximum relative standard deviation of drug content between single tablets of 6%. Moreover, the domains are large enough to provide deterrence against sniffing, i.e. access to the abuse-relevant drug by inhalation of the domains, and injection, i.e. intradermal, subdermal, subcutaneous, intramuscular, intraveneous or other forms of injection. While in view of the above variations of the number of domains will be determined depending on the parameters of the abuse-deterrent drug formulation, a plurality of discrete domains in representative drug formulations corresponds to a range of about 10 to about 1000, in particular about 50 to about 750, such as about 100 to about 500 domains per dosage unit. For example, each one dosage unit of a given drug formulation may contain about 100, about 150, about 200, about 250, about 300, or about 350 domains. The size is greater than 100 micrometers. In particular, the sizes may range from about 100 micrometers to about 1100 micrometers or from about 100 micrometers to about 1000 micrometers. In one aspect, for use in abuse deterrence, the particles preferably have a size of more than 500 micrometers.

In one aspect, at least 90% by weight of the domains have a particle size within a range from 100 to 1000 micrometers, or at least 90% by weight of the domains have a particle size within a range from 300 to 800 micrometers.

To those skilled in the art it is clear that, at a given amount of drug to be contained in a abuse-deterrent formulation of the invention, the size chosen for the particles will affect the number of domain-forming particles to be incorporated in said formulation, as outlined in the definitions above.

Particles constituting the discrete domains can be prepared by a variety of methods. In one aspect, the particles are prepared by melt extrusion to ensure high mechanical strength. For modifying the material properties, such as reducing brittleness, or aiding manufacturing processes, the domains may comprise additional components, such as plasticizers.

Process aids may be used to fine tune dynamic mechanical properties to maximize energy dissipation at the impact frequencies corresponding to commonly used methods of grinding and crushing.

The discrete domains of the abuse-deterrent formulation of the present invention may comprise one or more abuse-relevant drugs. The weight ratio of abuse-relevant drug and the polymer contained in the domains can range from about 50:50 to about 0.1 to 99.9, and may for example be about 40:60, about 30:70, about 20:80, about 10:90, about 5:95, about 2:98; about 1:99, about 0.75:99.25, about 0.5:99.5, about 0,25:99.75, or about 0.1:99.9.

The abuse-relevant drug can be embedded in the formulation in crystalline, amorphous or a combination thereof.

The discrete domains may contain one or more further active agents other than the abuse-relevant drug(s). In particular, the one or more active agents will be chosen to exert a synergistic effect in combination with the one or more abuse-relevant drugs. In one aspect, the active agent may be selected from the group consisting of salicylates, anthranilic acid derivatives, arylacetic acid derivatives, arylpropionic acid derivatives, oxicames and pyrazolideindiones. An example for particularly useful further active agent is paracetamole.

The discrete domains are surrounded by and embedded in a pharmaceutically acceptable matrix. The matrix provides a template for incorporating the abuse resistant domains. Moreover, the matrix can advantageously contain at least one further active agent in amorphous or crystalline form. The matrix can be designed for a range of release rates from immediate to extended release of up to 24 hours.

In one aspect, said further active agent contained in the matrix is not an abuse-relevant drug. In particular, the the one or more active agents will be chosen to exert a synergistic effect in combination with the one or more abuse-relevant drugs.

Particular examples of abuse-deterrent drug formulations comprise hydrocodone as abuse-relevant drug and paracetamole as further active agent. Specific examples relate to abuse-deterrent drug formulations comprising 10 mg hydrocodone bitartrate and 650 mg paracetamole per tablet, or 15 mg hydrocodon bitartrate and 650 mg paracetamole per tablet

When the drug formulations comprise one or more further active agents, said ingredient(s) may be present in the pharmaceutically active matrix, or in the discrete domains, or both in the matrix and the domains, thereby advantageously increasing the storage capacity of the abuse-deterrent drug formulation for the further ingredient by making use of the storage capacity of the discrete domains.

The matrix may be composed of any powder composition of sufficient adhesive or cohesive properties to form, after compression, a hard, strong dosage form. A binder is usually required to bond the powder particles together due to the poor cohesive properties of most powders.

Typically, the binder is at least one pharmaceutically acceptable polymeric binder. Pharmaceutically acceptable polymeric binders are known in the art of drug formulation. Examples of pharmaceutically acceptable polymers are those selected from cellulose esters and cellulose ethers, in particular methylcellulose and ethylcellulose, hydroxyalkylcelluloses, in particular hydroxypropylcellulose,

hydroxyalkylalkylcelluloses, in particular hydroxypropylmethylcellulose, cellulose phthalates or succinates, in particular cellulose acetate phthalate and hydroxypropylmethylcellulose phthalate, hydroxypropylmethylcellulose succinate or hydroxypropylmethylcellulose acetate succinate, starch and modified starch, homopolymers and copolymers of N-vinyl lactams, especially homopolymers and copolymers of N-vinyl pyrrolidone, e.g. polyvinylpyrrolidone (PVP), copolymers of N-vinyl pyrrolidone and vinyl acetate or vinyl propionate,
polyvinyl alcohol-polyethylene glycol-graft copolymers (available as Kollicoat® IR from BASF AG, Ludwigshafen, Germany); polyacrylates and polymethacrylates such as methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates), polyacrylamides, vinyl acetate polymers such as copolymers of vinyl acetate and crotonic acid, partially hydrolyzed polyvinyl acetate (also referred to as partially saponified “polyvinyl alcohol”), polyvinyl alcohol, oligo- and polysaccharides such as carrageenans, galactomannans and xanthan gum, and mixtures of one or more of the above or mentioned pharmaceutically acceptable polymeres.

Hydroxypropyl methylcellulose is an example of a cellulose ether. Hydroxpropyl methyicellulose Is available under the brand name METHOCEL E (methyl D. S. about 1.9, hydroxypropyi molar substitution about 0.23), METHOCEL F (methyl D. S. about 1.8, hydroxypropyi molar substitution about 0.13), and METHO- CEL K (methyl D. S. about 1.4, hydroxypropyi molar substitution about 0.21). METHOCEL E and METHOCEL K are examples of hydroxpropyl methylcelluloses for use in the present invention.

Examples of (meth)acrylic polymers are ionic (meth)acrylic polymers, in particular cationic (meth)acrylic polymers, such as those comprising quaternary ammonium groups. Suitable (meth)acrylic polymers are commercially available from Rohm Pharma under the Tradename Eudragit, preferably Eudragit RL and Eudragit RS. Eudragit RL and Eudragit RS are copolymers of acrylic and methacrylic esters with a low content of quaternary ammonium groups, the molar ratio of ammonium groups to the remaining neutral (meth)acrylic esters being 1 :20 in Eudragit RL and 1:40 in Eudragit RS. The mean molecular weight is about 150,000.

Other examples for (meth)acrylic polymers are anionic (meth)acrylic polymers. EUDRAGIT® L 100 and EUDRAGIT® S 100 are anionic copolymers based on methacrylic acid and methyl methacrylate. The ratio of the free carboxyl groups to the ester groups is approx. 1:1 in EUDRAGIT® L 100 and approx. 1:2 in EUDRAGIT® S 100. The average molecular weight is approx. 135,000.

In other aspects, the matrix comprises a combination of (i) at least one cellulose esters and cellulose ethers, and (ii) at least one (meth)acrylic polymer. The weight ration of (i) to (ii) may range from about 10:1 to about 1:1, from about 8:1 to about 2:1.

Those skilled in the art will appreciate that the domains and/or the matrix of the abuse-deterrent drug formulations may comprise one or more additives, which are useful for modifying the properties of the drug formulation, e.g. release profile of the drugs or further ingredients, or facilitate the preparation of the abuse-deterrent drug formulations as described below, e.g. by lowering glass transition temperatures during extrusion processes.

Examples for additives comprise flow regulators, disintegrants. Likewise, for fine-tuning the release of the abuse-relevant drug from the domains, the particles underlying the domains may contain disintegrants. In such cases amounts of disintegrant are chosen, which essentially do not impair abuse-deterrent features of the particles, such as crushing resistance. Corresponding amount can routinely be determined by those skilled in the art. Suitable disintegrants are crosslinked polymers such as crosslinked polyvinyl pyrrolidone and crosslinked sodium carboxymethyl cellulose. Suitable bulking agents (also referred to as “fillers”) are selected from lactose, calcium hydrogenphosphate, microcrystalline cellulose (Avicel®), magnesium oxide, potato or corn starch, isomalt, polyvinyl alcohol.

Suitable flow regulators are selected from highly dispersed silica (Aerosil®), and animal or vegetable fats or waxes. Flow regulators may advantageously be used in the matrix, particularly when matrix components in powder form are used for embedding the particles in a matrix by compression, but may also be used as component of extrudates for preparing the particles or the matrix, or as components of mixtures for preparing particles by methods such as spray drying or wet granulation.

Lubricants can be used when the preparation of the abuse-deterrent drug formulations comprises a compression step. The lubricants reduce the friction between the die wall and the tablet formulation during the compression and thus facilitate the ejection of the tablet from the die. Suitable lubricants are selected from polyethylene glycol (e.g., having a Mw of from 1000 to 6000), magnesium and calcium stearates, sodium stearyl fumarate, talc, and the like.

Various other additives may be used in the matrix or in the particles, for example dyes such as azo dyes, organic or inorganic pigments such as aluminium oxide or titanium dioxide, or dyes of natural origin; stabilizers such as antioxidants, light stabilizers, radical scavengers, or stabilizers against microbial attack.

Polyethylene oxides are known to need a small amount of antioxidant in order to have sufficient storage stability. For example, the commercial available Polyox ® contains a small amount of butyl hydroxy toluene (BHT). In order to have a suitable stabilization of the polyethylene oxide during storage in the final formulation it may be needed to add additional antioxidant to the formulation. Examples of antioxidants are those which are listed in common pharmacopoeas like e.g. USP/NF and/or Pharm. Eur.

The abuse-deterrent drug formulations of the invention can be provided in unit dosage form, for example in the form of tablets, pills or suppositories, suitable for administration to a subject. The administration may be oral, by implantation into tissues, e.g. subcutaneous, or anal. The amounts of abuse-relevant drug or further active agent will depend on the respective drugs and ingredients, the patient group to be treated in dependence of factors such as age, gender, severity of the treated condition, the intended frequency of administration. Such factors are known to those skilled in the art and can be determined appropriately. While therefore exact amounts are determined in each case, examples for generic ranges of amounts of abuse-relevant drug or further active agent are from 0.01 mg to 5000 mg, from 0.1 mg to 500 mg, or from 1 mg to 50 mg.

In order to facilitate the intake of an abuse-deterent drug formulation by a subject, it is advantageous to provide it as a dosage form with an appropriate shape. Large tablets that can be swallowed comfortably are therefore elongated rather than round in shape.

A film coat on the tablet further contributes to the ease with which it can be swallowed. A film coat also improves taste and provides an elegant appearance. If desired, the film coat may be an enteric coat. The film coat usually includes a polymeric film-forming material such as hydroxypropyl methylcellulose, hydroxypropyl cellulose, and acrylate or methacrylate copolymers. Besides a film-forming polymer, the film coat may further comprise a plasticizer, e.g. polyethylene glycol, a surfactant, e.g. a Tween® type, and optionally a pigment, e.g. titanium dioxide or iron oxides. The film-coating may also comprise talc as anti-adhesive. The film coat usually accounts for less than about 5% by weight of the dosage form.

In some cases it may be needed to obtain a very fast onset of the drug release of the non-abuse-relevant drug of the final formulation. In this case the film coat may contain a part of the total non-abuse-relevant drug amount which is present in the final tablet. The fast dissolving coat layer is able to release this portion of the non-abuse-relevant drug much faster than the rest of the non-abuse-relevant drug which is present in the tablet core. By this way a biphasic drug release can be achieved (“fast/slow”). The amount of drug being present in the coat compared to the total drug amount in the final tablet is in the range of 0 to 35% w/w, 0 to 20% w/w mg and 0 to 15% w/w.

The present invention further relates to methods for preparing abuse-deterring drug formulations, in particular those drug formulations described above. The methods comprise in a first step a) the manufacturing of particles, which comprise a fracture toughness-imparting polymer and at least one abuse-relevant drug, and in a second step b) comprise embedding a plurality of said particles in a pharmaceutically acceptable matrix in a unit dosage form. By embedding the particles in the pharmaceutically acceptable matrix, the particles form the discrete domains as defined above within the matrix.

The particles, which comprise a fracture toughness-imparting polymer and at least one abuse-relevant drug, may be prepared by a variety of methods. For example, the particles can be prepared by hot-melt extrusion. Hot-melt extrusion processes involve the formation of a formable composition, wherein the composition comprises the comprising the fracture toughness-imparting polymer and the abuse-relevant drug; and forcing the composition through at least one orifice. The extruded solid is sized to form particles. One or more further ingredients or additives may be also comprised in the formable composition.

For the hot-melt extrusion process extruders or kneaders may be used. Suitable extruders include single screw extruders, intermeshing screw extruders or else multiscrew extruders, or twin screw extruders, which can be corotating or counterrotating and, optionally, equipped with kneading disks or other screw elements for mixing or dispersing the melt. It will be appreciated that the working temperatures will also be determined by the kind of extruder or the kind of configuration within the extruder used. Part of the energy needed to melt, mix and dissolve the components in the extruder can be provided by heating elements. However, the friction and shearing of the material in the extruder may also provide a substantial amount of energy to the mixture and aid in the formation of a homogeneous melt of the components.

The hot-melt extrusion process employed in some embodiments of the invention is conducted at an elevated temperature, i.e. the heating zone(s) of the extruder is above room temperature. It is important to select an operating temperature range that will minimize the degradation or decomposition of the therapeutic compound during processing. The operating temperature range is generally in the range of from about 60° C. to about 220° C., from about 70° C. to about 180° C. and from about 80° C. to about 160° C. as determined by the setting for the extruder heating zone(s).

In some embodiments of the invention, the hot-melt extrusion may be conducted employing a slurry, solid, suspension, liquid, powdered or other such feed comprising the fracture toughness-imparting polymer and an abuse-relevant drug. Dry feed is advantageously employed in the process of the present invention. Liquid or semisolid components of the formulation composition can be dosed into the extruder at room temperature or at higher temperatures by a suitable pump.

The hot-melt extrusion process is generally described as follows. An effective amount of a powdered abuse-relevant drug is mixed with a fracture toughness-imparting polymer, and in some embodiments, with a plasticizer. Other components may be added in the various embodiments of the invention. In some embodiments. The mixture is then placed in the extruder hopper and passed through the heated area of the extruder at a temperature which will melt or soften the fracture toughness-imparting polymer and/or plasticizer, if present, to form a matrix throughout which the abuse-relevant drug is dispersed. The molten or softened mixture then exits via a die, or other such element, at which time, the mixture (now called the extrudate) begins to harden. Since the extrudate is still warm or hot upon exiting the die, it may be easily shaped, molded, chopped, ground, molded, sphegonized into beads, cut into strands, tableted or otherwise processed to the desired physical form.

One method for sizing the particles is hot-spheronization, which yields spheronized particles. The particles can be prepared by hot spheronization. In general, a spheronizers comprises a chamber with a spinning disk, the surface of which shows a pattern of lines or groves. The extrudate is introduced into the chamber and by contact with the spinning disk is disintegrated into small fragments, hurled towards the walls of the chamber and repeatedly reflected back and forth. The contact with the spinning disk, the chamber walls and the collisions between extrudate particles finally causes the final sphericals shape of the extrudate particles.

Another process is to force the drug-containing extrudate out of the extruder die which contains small holes. The spaghetti-like extrudate is cut directly in front of the die by a fast-rotating knife. This so-called “hot-cutting” process enables the formation of small cylindrical melt pieces which are still in the molten state after cutting. These melt pieces solidify at room temperature following the cutting step and then can be used for the formulations according to the present invention. If spherical pieces are needed these cylindrical pieces can be rounded in a separate step. In one aspect, this rounding is performed when the pieces are still in the molten state.

The particles can also be prepared by spray-drying, a method well-known in the art. In spray-drying, the fracture toughness-imparting polymer and the abuse-relevant drug are dissolved in a common solvent. The liquid is then suspended in a gas flow, e.g., air, e.g., the liquid is converted into a fog-like mist (atomized), providing a large surface area. The atomized liquid is exposed to a flow of hot gas in a drying chamber. The moisture evaporates quickly and the solids are recovered as a powder consisting of fine, hollow spherical particles. Gas inlet temperatures of up to 250° C. or even higher may be used, due to the evaporation the gas temperature drops very rapidly to a temperature of about 30 to 150° C. (outlet temperature of the gas). Decomposition or modification of the abuse-relevant drug or the further active agent(s) is avoided or minimized by choosing temperatures low enough or by exposure times short enough to avoid such adverse effects.

Wet granulation is a further process that can be used to produce particles containing the fracture toughness-imparting polymer and the abuse-relevant drug. The procedure consists of mixing the powders in a suitable blender followed by adding a granulating solution under shear to the mixed powders to obtain a granulation The damp mass is then screened through a suitable screen and dried by tray dying or fluidized bed drying. Alternately, the wet mass may be dried and passed through a mill

Methods for embedding particles comprise blending said particles with a compressible powder to yield a powder blend and compressing the powder blend into a unit dosage form. Blending and compression procedures are known to those skilled in the art. The procedures can readily be adapted to chosen combinations of particles and compressible powder. Compression, also referred to as compacting, means a process whereby a powder mass comprising the particles is densified under high pressure in order to obtain a compact with low porosity, e.g. a tablet. Compression of the powder mass is usually done in a tablet press, more specifically in a steel die between two moving punches.

Tablet presses are well-known in the art as devices for compressing powders and/or particulates into tablets of essentially uniform weight and size. For this purpose dies between two movable punches are filled with the powders/particulates to be compressed. Pressing the punches against each other compresses the powders/particulates into a solid tablet. In one aspect, rotary tablet presses are used for preparing the dosage forms of the abuse-deterrent drug formulations of the invention.

The powders may have been subjected to wet granulation, or dry granulation as needed.

In one aspect, the constituents of the pharmaceutically acceptable matrix are melt-processed, for example hot-melt extruded, and the extrudate is milled or ground to a powder. The powder is then blended with the particles containing the fracture toughness-imparting polymer and the abuse-relevant drug and compressed, yielding the particles forming domains in the solid matrix.

A further example for embedding the plurality of particles in a pharmaceutically acceptable matrix comprises liquefying the corresponding matrix composition by heating, dispersing said particles in the liquefied matrix composition, shaping the liquefied composition into a unit dosage form, and solidifying the composition.

### III. Other Drug Formulations

A. Formulations Comprising an Active Agent, an Abuse-Relevant Drug or a Combination of an Active Agent and an Abuse-Relevant Drug in Substantially Crystalline Form

In another embodiment, the present invention relates to a formulation comprising at least one active agent, at least one abuse-relevant drug or a combination of an active agent and an abuse-relevant drug that is in substantially crystalline form and is uniformly dispersed in a pharmaceutically acceptable matrix containing at least one polymer having a high fracture toughness. The formulations of the present invention, in one aspect, can be used as abuse-deterrent formulations or as tamper resistant formulations.

More specifically, the pharmaceutically acceptable matrix contains at least one active agent, at least one abuse-relevant drug or a combination of at least one active agent and at least one abuse-relevant drug that is in substantially crystalline form. In addition, the matrix also contains at least one polymer. In one aspect, the at least one polymer is a semi-crystalline polymer, a high loss modulus polymer or a combination of a semi-crystalline polymer and a high loss modulus polymer. Optionally, the matrix may also comprise at least one plasticizer. When the pharmaceutically acceptable matrix contains PEO, the formulations of the present invention are shapeable at room temperature unlike other formulations known in the art which have to re-heated to be re-shaped Impurities are introduced into such formulations when they are re-heated for the purposes of re-shaping.

The active agent, abuse-relevant drug or combination of active agent and abuse-relevant drug used in the formulation, in addition to being in substantially crystalline form also does not dissolved in the polymer. In one aspect, the active agent and/or abuse-deterrent agent has a melting point that is at least 170° C. (if no plasticizer is being used in the process). If a plasticizer is being used in the process, then active agent and/or abuse-deterrent agent preferably have a melting temperature of at least about 50° C. It is recognized that other active agents and/or abuse-relevant drugs may have different melting temperatures.

The matrix can be designed for a range of release rates from immediate to extended release of up to 24 hours.

As mentioned above, the pharmaceutically acceptable matrix of the formulations of the present invention may comprise one or more active agents, one or more abuse-relevant drugs or a combination of active agents and abuse-relevant drugs that are in substantially crystalline form. The weight ratio of active agents and/or abuse-relevant drug and polymer contained in the matrix can range from about 50:50 to about 0.1 to 99.9, and may for example be about 40:60, about 30:70, about 20:80, about 10:90, about 5:95, about 2:98; about 1:99, about 0.75:99.25, about 0.5:99.5, about 0,25:99.75, or about 0.1:99.9.

Those skilled in the art will appreciate that the the matrix of the formulations may comprise one or more additives, which are useful for modifying the properties of the drug formulation, e.g. release profile of the drugs or further ingredients, or facilitate the preparation of the formulations as described below, e.g. by lowering glass transition temperatures during extrusion processes.

Examples for additives comprise flow regulators, disintegrants. Likewise, for fine-tuning the release of the abuse-relevant drug, the matrix may contain disintegrants. In such cases amounts of disintegrant are chosen, which essentially do not impair abuse-deterrent features of the particles, such as crushing resistance. Corresponding amount can routinely be determined by those skilled in the art. Suitable disintegrants are crosslinked polymers such as crosslinked polyvinyl pyrrolidone and crosslinked sodium carboxymethyl cellulose. Suitable bulking agents (also referred to as “fillers”) are selected from lactose, calcium hydrogenphosphate, microcrystalline cellulose (Avicel®), magnesium oxide, potato or corn starch, isomalt, polyvinyl alcohol.

Suitable flow regulators are selected from highly dispersed silica (Aerosil®), and animal or vegetable fats or waxes. Flow regulators may advantageously be used in the matrix, particularly to achieve a uniform blend in the blending step prior to extrusion.

Lubricants can be used when the preparation of the formulations comprise a compression step. The lubricants reduce the friction between the die wall and the tablet formulation during the compression and thus facilitate the ejection of the tablet from the die. Suitable lubricants are selected from polyethylene glycol (e.g., having a Mw of from 1000 to 6000), magnesium and calcium stearates, sodium stearyl fumarate, talc, and the like.

Various other additives may be used in the matrix or in the particles, for example dyes such as azo dyes, organic or inorganic pigments such as aluminium oxide or titanium dioxide, or dyes of natural origin; stabilizers such as antioxidants, light stabilizers, radical scavengers, or stabilizers against microbial attack.

Polyethylene oxides are known to need a small amount of antioxidant in order to have sufficient storage stability. For example, the commercial available Polyox® contains a small amount of butyl hydroxy toluene (BHT). In order to have a suitable stabilization of the polyethylene oxide during storage in the final formulation it may be needed to add additional antioxidant to the formulation. Examples of antioxidants are those which are listed in common pharmacopoeas like e.g. USP/NF and/or Pharm. Eur.

The formulations of the invention can be provided in unit dosage form, for example in the form of tablets, pills or suppositories, suitable for administration to a subject. The administration may be oral, by implantation into tissues, e.g. subcutaneous, or anal. The amounts of active agent, abuse-relevant drug or combination active agent and abuse-relevant drug will depend on the respective drugs and ingredients, the patient group to be treated in dependence of factors such as age, gender, severity of the treated condition, the intended frequency of administration. Such factors are known to those skilled in the art and can be determined appropriately. While therefore exact amounts are determined in each case, examples for generic ranges of amounts of active agent, abuse-relevant drug or combination of active agent and abuse-relevant drug are from 0.01 mg to 5000 mg, from 0.1 mg to 500 mg, or from 1 mg to 50 mg.

In order to facilitate the intake of a formulation by a subject, it is advantageous to provide it as a dosage form with an appropriate shape. Large tablets that can be swallowed comfortably are therefore elongated rather than round in shape.

A film coat on the tablet further contributes to the ease with which it can be swallowed. A film coat also improves taste and provides an elegant appearance. If desired, the film coat may be an enteric coat. The film coat usually includes a polymeric film-forming material such as hydroxypropyl methylcellulose, hydroxypropyl cellulose, and acrylate or methacrylate copolymers. Besides a film-forming polymer, the film coat may further comprise a plasticizer, e.g. polyethylene glycol, a surfactant, e.g. a Tween® type, and optionally a pigment, e.g. titanium dioxide or iron oxides. The film-coating may also comprise talc as anti-adhesive. The film coat usually accounts for less than about 5% by weight of the dosage form.

In some cases it may be needed to obtain a very fast onset of the drug release of the active agent of the final formulation. In this case the film coat may contain a part of the total active agent amount which is present in the final tablet. The fast dissolving coat layer is able to release the active agent, abuse-relevant drug or combination of active agent and abuse-relevant drug much faster than the rest of the active agent which is present in the tablet core. By this way a biphasic drug release can be achieved (“fast/slow”). The amount of drug being present in the coat compared to the total drug amount in the final tablet is in the range of 0 to 35% w/w, 0 to 20% w/w mg and 0 to 15% w/w.

The present invention further relates to methods for preparing formulations, in particular those drug formulations described above. The methods comprise in a first step the manufacturing the pharmaceutically acceptable matrix (comprising a semi-crystalline polymer or a fracture toughness-imparting polymer and at least one active agent, at least one abuse-relevant drug or a combination of at least one active agent and at least one abuse-deterrent drug and optionally, at least one plasticizer) in a unit dosage form.

Although the process referred to above has been called a hot-melt extrusion, other equivalents processes such as injection molding, hot dipping, melt casting and compression molding may be used.

For the hot-melt extrusion process extruders or kneaders may be used. Suitable extruders include single screw extruders, intermeshing screw extruders or else multiscrew extruders, or twin screw extruders, which can be corotating or counterrotating and, optionally, equipped with kneading disks or other screw elements for mixing or dispersing the melt. It will be appreciated that the working temperatures will also be determined by the kind of extruder or the kind of configuration within the extruder used. Part of the energy needed to melt, mix and dissolve the components in the extruder can be provided by heating elements. However, the friction and shearing of the material in the extruder may also provide a substantial amount of energy to the mixture and aid in the formation of a homogeneous melt of the components.

The hot-melt extrusion process employed in some embodiments of the invention is conducted at an elevated temperature, i.e. the heating zone(s) of the extruder is above room temperature. It is important to select an operating temperature range that will minimize the degradation or decomposition of the therapeutic compound during processing. The operating temperature range is generally in the range of from about 60° C. to about 220° C., from about 70° C. to about 180° C. and from about 80° C. to about 160° C. as determined by the setting for the extruder heating zone(s).

In some embodiments of the invention, the hot-melt extrusion may be conducted employing a slurry, solid, suspension, liquid, powdered or other such feed comprising the fracture toughness-imparting polymer and an abuse-relevant drug. Dry feed is advantageously employed in the process of the present invention. Liquid or semisolid components of the formulation composition can be dosed into the extruder at room temperature or at higher temperatures by a suitable pump.

The hot-melt extrusion process is generally described as follows. An effective amount of at least one active agent, at least one abuse-relevant drug or a combination of at least one active agent and at least one abuse-relevant drug is mixed with a fracture toughness-imparting polymer, and in some embodiments, with a plasticizer. Other components may be added in the various embodiments of the invention. In some embodiments the mixture is then placed in the extruder hopper and passed through the heated area of the extruder at a temperature which will melt or soften the fracture toughness-imparting polymer and/or plasticizer, if present, to form a matrix throughout which the active agent and/or abuse-relevant drug is dispersed. The molten or softened mixture then exits via a die, or other such element, at which time, the mixture (now called the extrudate) begins to harden. Since the extrudate is still warm or hot upon exiting the die, it may be easily shaped, molded, chopped, ground, molded, sphegonized into beads, cut into strands, tableted or otherwise processed to the desired physical form.

One method for sizing the particles is hot-spheronization, which yields spheronized particles. The particles can be prepared by hot spheronization. In general, a spheronizers comprises a chamber with a spinning disk, the surface of which shows a pattern of lines or groves. The extrudate is introduced into the chamber and by contact with the spinning disk is disintegrated into small fragments, hurled towards the walls of the chamber and repeatedly reflected back and forth. The contact with the spinning disk, the chamber walls and the collisions between extrudate particles finally causes the final sphericals shape of the extrudate particles.

Another process is to force the drug-containing extrudate out of the extruder die which contains small holes. The spaghetti-like extrudate is cut directly in front of the die by a fast-rotating knife. This so-called “hot-cutting” process enables the formation of small cylindrical melt pieces which are still in the molten state after cutting. These melt pieces solidify at room temperature following the cutting step and then can be used for the formulations according to the present invention. If spherical pieces are needed these cylindrical pieces can be rounded in a separate step. In one aspect, this rounding is performed when the pieces are still in the molten state. If the melt pieces, immediately, or after moderate cooling, have sufficient plastic deformability at ambient temperature, the pieces can be compressed into a monolithic tablet. If the melt pieces do not have sufficient deformability at ambient temperature, the melt pieces may be rendered plastically deformable by heating prior to compression, such as by heating or re-heating prior to charging into a die cavity.

B. Formulations Comprising a Plurality of Discrete Mechanically Reinforcing Particles Uniformly Dispersed in a Pharmaceutically Active Matrix

In yet another embodiment, the present invention relates to a formulation comprising a plurality of discrete mechanically reinforcing particles uniformly dispersed in a pharmaceutically acceptable matrix, wherein the matrix comprises at least one polymer having a high fracture toughness. The discrete mechanically reinforcing particles comprise at least one filler, at least one fiber or a combination of at least one filler and at least one fiber. An example of a filler that can be used in the discrete mechanically reinforcing domains is dicalcium phosphate (DCP). An example of a fiber that can be used is a fiber made from a cellulosic excipient or any other pharmaceutically acceptable fibrous material. When the pharmaceutically acceptable matrix contains PEO, the formulations of the present invention are shapeable at room temperature unlike other formulations known in the art which have to re-heated to be re-shaped. There is always a risk that impurities can be introduced into such formulations when they are re-heated for the purposes of re-shaping.

The formulations of the present invention, in one aspect, can be used as abuse-deterrent formulations or as tamper resistant formulations.

The “plurality” of mechanically reinforcing particles as used in reference to the formulations of the present invention refers to at least two particles. The particles constituting the discrete domains can be prepared by a variety of methods.

The discrete particles are surrounded by and embedded in a pharmaceutically acceptable matrix. The matrix contains at least one active agent, at least one abuse-relevant drug or a combination of at least one active agent and at least one abuse-deterrent drug. In addition, the matrix also contains at least one polymer. In one aspect, the at least one polymer is a semi-crystalline polymer, a high loss modulus polymer or a combination of a semi-crystalline polymer and a high loss modulus polymer. Optionally, the pharmaceutically acceptable matrix can further comprise at least one plasticizer. The matrix can be designed for a range of release rates from immediate to extended release of up to 24 hours.

The matrix of the formulations of the present invention may comprise one or more active agents, one or more abuse-relevant drugs or a combination of active agents and abuse-relevant drugs. The weight ratio of active agent and/or abuse-relevant drug and polymer contained in the matrix can range from about 50:50 to about 0.1 to 99.9, and may for example be about 40:60, about 30:70, about 20:80, about 10:90, about 5:95, about 2:98; about 1:99, about 0.75:99.25, about 0.5:99.5, about 0,25:99.75, or about 0.1:99.9.

Those skilled in the art will appreciate that the matrix of the formulations may comprise one or more additives, which are useful for modifying the properties of the drug formulation, e.g. release profile of the drugs or further ingredients, or facilitate the preparation of the formulations as described below, e.g. by lowering glass transition temperatures during extrusion processes.

Examples of additives include flow regulators and disintegrants. Likewise, for fine-tuning the release of the abuse-relevant drug from the matrix can be achieved by using a distintegrant. The amount of distintegrant can routinely be determined by those skilled in the art. Suitable disintegrants are crosslinked polymers such as crosslinked polyvinyl pyrrolidone and crosslinked sodium carboxymethyl cellulose.

Suitable flow regulators are selected from highly dispersed silica (Aerosil®), and animal or vegetable fats or waxes. Flow regulators may advantageously be used in the matrix, to achieve uniform blending prior to extrusion.

Lubricants can be used when the preparation of the formulations comprises a compression step. The lubricants reduce the friction between the die wall and the tablet formulation during the compression and thus facilitate the ejection of the tablet from the die. Suitable lubricants are selected from polyethylene glycol (e.g., having a Mw of from 1000 to 6000), magnesium and calcium stearates, sodium stearyl fumarate, talc, and the like.

Various other additives may be used in the matrix, for example dyes such as azo dyes, organic or inorganic pigments such as aluminium oxide or titanium dioxide, or dyes of natural origin; stabilizers such as antioxidants, light stabilizers, radical scavengers, or stabilizers against microbial attack.

Polyethylene oxides are known to need a small amount of antioxidant in order to have sufficient storage stability. For example, the commercial available Polyox® contains a small amount of butyl hydroxy toluene (BHT). In order to have a suitable stabilization of the polyethylene oxide during storage in the final formulation it may be needed to add additional antioxidant to the formulation. Examples of antioxidants are those which are listed in common pharmacopoeas like e.g. USP/NF and/or Pharm. Eur.

The formulations of the invention can be provided in unit dosage form, for example in the form of tablets, pills or suppositories, suitable for administration to a subject. The administration may be oral, by implantation into tissues, e.g. subcutaneous, or anal. The amounts of at least one active agent, at least one abuse-relevant drug or a combination of at least one active agent and at least one abuse-relevant drug will depend on the respective drugs and ingredients, the patient group to be treated in dependence of factors such as age, gender, severity of the treated condition, the intended frequency of administration. Such factors are known to those skilled in the art and can be determined appropriately. While therefore exact amounts are determined in each case, examples for generic ranges of amounts of active agent, abuse-relevant drug or active agent and abuse-relevant drug are from 0.01 mg to 5000 mg, from 0.1 mg to 500 mg, or from 1 mg to 50 mg.

In order to facilitate the intake of a formulation by a subject, it is advantageous to provide it as a dosage form with an appropriate shape. Large tablets that can be swallowed comfortably are therefore elongated rather than round in shape.

A film coat on the tablet further contributes to the ease with which it can be swallowed. A film coat also improves taste and provides an elegant appearance. If desired, the film coat may be an enteric coat. The film coat usually includes a polymeric film-forming material such as hydroxypropyl methylcellulose, hydroxypropyl cellulose, and acrylate or methacrylate copolymers. Besides a film-forming polymer, the film coat may further comprise a plasticizer, e.g. polyethylene glycol, a surfactant, e.g. a Tween® type, and optionally a pigment, e.g. titanium dioxide or iron oxides. The film-coating may also comprise talc as anti-adhesive. The film coat usually accounts for less than about 5% by weight of the dosage form.

In some cases it may be needed to obtain a very fast onset of the drug release of the active agent, abuse-relevant drug or combination of active agent and abuse-relevant drug of the final formulation. In this case the film coat may contain a part of the total active agent amount which is present in the final tablet. The fast dissolving coat layer is able to release the active agent much faster than the rest of the active agent which is present in the tablet core. By this way a biphasic drug release can be achieved (“fast/slow”). The amount of drug being present in the coat compared to the total drug amount in the final tablet is in the range of 0 to 35% w/w, 0 to 20% w/w mg and 0 to 15% w/w.

The present invention further relates to methods for preparing formulations, in particular those drug formulations described above. The methods comprise in a first step a) the manufacturing of particles, which comprise a filler (such as dicalcium phosphate) or a fiber (such as s a cellulosic excipient or any other pharmaceutically acceptable fibrous material) and in a second step b) comprise embedding a plurality of said particles in a pharmaceutically acceptable matrix (comprising a semi-crystalline polymer or a fracture toughness-imparting polymer and at least one active agent, at least one abuse-relevant drug or a combination of at least one active agent and at least one abuse-deterrent drug and optionally, at least one plasticizer) in a unit dosage form. By embedding the particles in the pharmaceutically acceptable matrix, the particles form the discrete domains as defined above within the matrix.

The matrix may be prepared by a variety of methods. For example, the matrix can be prepared by hot-melt extrusion. Hot-melt extrusion processes involve the formation of a formable composition, wherein the composition comprises the comprising the filler or fiber and forcing the composition through at least one orifice. The extruded solid is sized to form particles. One or more further ingredients or additives may be also comprised in the formable composition.

Although the process referred to above has been called a hot-melt extrusion, other equivalents processes such as injection molding, hot dipping, melt casting and compression molding may be used.

For the hot-melt extrusion process extruders or kneaders may be used. Suitable extruders include single screw extruders, intermeshing screw extruders or else multiscrew extruders, or twin screw extruders, which can be corotating or counterrotating and, optionally, equipped with kneading disks or other screw elements for mixing or dispersing the melt. It will be appreciated that the working temperatures will also be determined by the kind of extruder or the kind of configuration within the extruder used. Part of the energy needed to melt, mix and dissolve the components in the extruder can be provided by heating elements. However, the friction and shearing of the material in the extruder may also provide a substantial amount of energy to the mixture and aid in the formation of a homogeneous melt of the components.

The hot-melt extrusion process employed in some embodiments of the invention is conducted at an elevated temperature, i.e. the heating zone(s) of the extruder is above room temperature. It is important to select an operating temperature range that will minimize the degradation or decomposition of the therapeutic compound during processing. The operating temperature range is generally in the range of from about 60° C. to about 220° C., from about 70° C. to about 180° C. and from about 80° C. to about 160° C. as determined by the setting for the extruder heating zone(s).

In some embodiments of the invention, the hot-melt extrusion may be conducted employing a slurry, solid, suspension, liquid, powdered or other such feed comprising the fracture toughness-imparting polymer and an abuse-relevant drug. Dry feed is advantageously employed in the process of the present invention. Liquid or semisolid components of the formulation composition can be dosed into the extruder at room temperature or at higher temperatures by a suitable pump.

The hot-melt extrusion process is generally described as follows. An effective amount of at least one active agent, at least one abuse-relevant drug or a combination of at least one active agent and at least one abuse-relevant drug is mixed with a fracture toughness-imparting polymer, and in some embodiments, with a plasticizer. Other components may be added in the various embodiments of the invention. In some embodiments the mixture is then placed in the extruder hopper and passed through the heated area of the extruder at a temperature which will melt or soften the fracture toughness-imparting polymer and/or plasticizer, if present, to form a matrix throughout which the active agent and/or abuse-relevant drug is dispersed. The molten or softened mixture then exits via a die, or other such element, at which time, the mixture (now called the extrudate) begins to harden. Since the extrudate is still warm or hot upon exiting the die, it may be easily shaped, molded, chopped, ground, molded, spheronized into beads, cut into strands, tableted or otherwise processed to the desired physical form.

One method for sizing the particles is hot-spheronization, which yields spheronized particles. The particles can be prepared by hot spheronization. In general, a spheronizers comprises a chamber with a spinning disk, the surface of which shows a pattern of lines or groves. The extrudate is introduced into the chamber and by contact with the spinning disk is disintegrated into small fragments, hurled towards the walls of the chamber and repeatedly reflected back and forth. The contact with the spinning disk, the chamber walls and the collisions between extrudate particles finally causes the final sphericals shape of the extrudate particles.

Another process is to force the drug-containing extrudate out of the extruder die which contains small holes. The spaghetti-like extrudate is cut directly in front of the die by a fast-rotating knife. This so-called “hot-cutting” process enables the formation of small cylindrical melt pieces which are still in the molten state after cutting. These melt pieces solidify at room temperature following the cutting step and then can be used for the formulations according to the present invention. If spherical pieces are needed these cylindrical pieces can be rounded in a separate step. In one aspect, this rounding is performed when the pieces are still in the molten state.

Methods for embedding particles comprise blending said reinforcing particles with a compressible powder to yield a powder blend and compressing the powder blend into a unit dosage form. Blending and compression procedures are known to those skilled in the art. The procedures can readily be adapted to chosen combinations of particles and compressible powder. Compression, also referred to as compacting, means a process whereby a powder mass comprising the particles is densified under high pressure in order to obtain a compact with low porosity, e.g. a tablet. Compression of the powder mass is usually done in a tablet press, more specifically in a steel die between two moving punches.

Tablet presses are well-known in the art as devices for compressing powders and/or particulates into tablets of essentially uniform weight and size. For this purpose dies between two movable punches are filled with the powders/particulates to be compressed. Pressing the punches against each other compresses the powders/particulates into a solid tablet. In one aspect, rotary tablet presses are used for preparing the dosage forms of the formulations of the invention.

The powders may have been subjected to wet granulation, or dry granulation as needed.

By way of example and not of limitation, examples of the present invention shall now be given.
APAP: N-acetyl-p-aminophenol
BHT: Butylhydroxytoluene
Compap 90™: Acetaminophen USP 90% anhydrous basis
Eud L 100-55: Eudragit® L 100-55
HCB: hydrocodone bitratrate (4,5a-Epoxy-3-methoxy-17-methylmorphinan-6-one bitartrate)
HPC: hydroxypropyl cellulose
HPMC: hydroxypropyl methylcellulose (hypromellose)
PEO: poly(ethylene oxide)

### EXAMPLE 1

### Formulation of Section II

This example exemplifies a composite formulation with 5% w/w Hydrocodone Bitartrate drug in a PEO phase. The PEO phase was dispersed in a rate controlling blend of HPMC and Eudragit L 100-55 and shaped into tablets by direct compression.

PEO domains were manufactured by melt extruding a blend of the components given in Table 1, followed by add pelletization. The PEO phase is assumed to be essentially spherical with an average equivalent diameter of about 1000 microns.

TABLE 1
PEO phase

| | mg | % w/w |
|---|---|---|
| APAP | 135.4 | 67.7 |
| HCB | 10.0 | 5.0 |
| Polyox N10 | 24.0 | 12.0 |
| Polyox WSR 301 | 24.0 | 12.0 |
| Titanium dioxide | 4.0 | 2.0 |
| BHT | 0.6 | 0.3 |
| Collodial anhydrous silica | 2.0 | 1.0 |
| | 200.0 | 100.0 |

The PEO pellets were uniformly blended with the ingredients given in Table 2 below and then compressed into tablets using a conventional rotary press.

TABLE 2
Drug formulation - Formulation Made Action to Section II

| | mg | % w/w |
|---|---|---|
| Compap 90 (APAP) | 571.8 | 55.3 |
| Spheronized PEO phase | 200.0 | 10.3 |
| HPMC K100LV | 172.0 | 16.6 |
| HPC | 10.0 | 1.0 |
| Eud L 100-55 | 30.0 | 2.9 |
| Lactose | 40.0 | 3.9 |
| SiO₂ | 5.0 | 0.5 |
| Magnesium stearate | 5.0 | 0.5 |
| | 1033.8 | 100.0 |

In the tablets thus obtained, the first rate controlling step is primarily the swelling/erosion of HPMC-Eudragit, followed by the swelling/erosion of the PEO domains.

### EXAMPLE 2

This example describes how to make a formulation described in Section IIIA.

### Blending Procedure:

1) Weigh the materials listed in Table 3.
2) Dispense materials listed in Table 3 except APAP and colloidial silica.
3) Mill BHT in a mortar and pestle.
4) Sieve BHT, TiO₂, Magnesium stearate and colloidal SiO₂ through a 30 mesh screen.
5) Mix by hand with a spatula.
6) Sieve both of the Poloxs' through a 30 mesh screen.
7) Blend above ingredients for 10 minutes.
8) Sieve the APAP through a 30 mesh screen.
9) Add the APAP and colloidial silica to the blend and blend another 10 minutes.

Once all the ingredients are blended, the mixture put mixture into hopper and melt-extruder. The mixture will go through three blocks before getting to the die. The first block is at a temperature of 110° C. The second block is at a 120° C. The third block is at 130° C. Once finished with the third block, the mixture moves to the die which is at 130° C. and the mixture is extruded. The extrudate then cools on a conveyor belt and then is cut to weight. The cut pieces are then put into a hydraulic press and tablets are formed.

TABLE 3
Formulation M-E - 10 (HCB)/650 (APAP)

| | mg | % |
|---|---|---|
| APAP dense powder | 520.0 | 68.3 |
| Hydrocodone bitartrate | 8.0 | 1.1 |
| Dow PEO WSR 301 LTO (4,000,000) | 78.0 | 10.3 |
| Dow PEO WSR N10 LTO (100,000) | 125.8 | 16.5 |
| Titanium dioxide (Titanium Dioxide | 15.3 | 2.0 |
| Spectrum USP/EP) | | |
| AO for PEO (BHT) | 2.6 | 0.3 |
| Lubricant: Magnesium Stearate | 3.8 | 0.5 |
| Glidant: Colloidal anhydrous silica (Aerosil 200) | 7.6 | 1.0 |
| Total Uncoated Tablet | 761.1 | 100.0 |
| Fraction plasticizer (/total polymer) - NA | | |
| Fraction low Mw polymer (/total polymer) - 0.62 | | |

### EXAMPLE 3

This example describes how to make a formulation described in Section IIIB The same procedure can be used for Formulations 17, 18 and 21 shown in Tables 4-6.

### Blending Procedure:

1) Weigh the materials listed in Table 3.
2) Dispense materials listed in Table 3 except APAP and colloidial silica.
3) Mill BHT in a mortar and pestle.
4) Sieve BHT, TiO₂, Magnesium stearate and colloidal SiO₂ through a 30 mesh screen.
5) Mix by hand with a spatula.
6) Sieve both of the Poloxs' through a 30 mesh screen.
7) Blend above ingredients for 10 minutes.
8) Sieve the APAP through a 30 mesh screen.
9) Add the APAP and colloidial silica to the blend and blend another 10 minutes.

Once all the ingredients are blended, the mixture put mixture into hopper and melt-extruder. The mixture will go through three blocks before getting to the die. The first block is at a temperature of 110° C. The second block is at a 120° C. The third block is at 130° C. Once finished with the third block, the mixture moves to the die which is at 130° C. and the mixture is extruded. The extrudate then cools on a conveyor belt and then is cut to weight. The cut pieces are then put into a hydraulic press and tablets are formed.

TABLE 4
Formulation 17 - 5 (HCB)/325 (APAP)

| | mg | % |
|---|---|---|
| APAP dense powder | 260.0 | 49.2 |
| Hydrocodone bitartrate | 4.0 | 0.8 |
| Dow PEO WSR 301 LTO (4,000,000) | 32.2 | 6.1 |
| Dow PEO 1105 (900,000) | 64.3 | 12.2 |
| Poloxamer 407 (Lutrol F127) | 33.8 | 6.4 |
| Titanium dioxide (Titanium Dioxide | 10.6 | 2.0 |
| Spectrum USP/EP) | | |
| AO for PEO (BHT) | 0.6 | 0.1 |
| Dicalcium Phosphate | 115.0 | 21.8 |
| Lubricant: Magnesium Stearate | 2.6 | 0.5 |
| Glidant: Colloidal anhydrous silica (Aerosil 200) | 5.3 | 1.0 |
| Total Uncoated Tablet | 528.3 | 100.0 |
| Fraction plasticizer (/total polymer) - 0.35 | | |
| Fraction low Mw polymer (/total polymer) - 0.67 | | |

TABLE 5
Formulation 18 - 5 (HCB)/325 (APAP)

| | mg | % |
|---|---|---|
| APAP dense powder | 520.0 | 69.2 |
| Hydrocodone bitartrate | 8.0 | 1.1 |
| Dow PEO WSR 301 LTO (4,000,000) | 45.0 | 6.0 |
| Dow PEO 1105 (900,000) | 90.0 | 12.0 |
| Poloxamer 407 (Lutrol F127) | 60.8 | 8.1 |
| Titanium dioxide (Titanium Dioxide | 15.0 | 2.0 |
| Spectrum USP/EP) | | |
| AO for PEO (BHT) | 1.5 | 0.2 |
| Lubricant: Magnesium Stearate | 3.8 | 0.5 |
| Glidant: Colloidal anhydrous silica (Aerosil 200) | 7.5 | 1.0 |
| Total Uncoated Tablet | 751.5 | 100.0 |
| Fraction plasticizer (/total polymer) - 0.45 | | |
| Fraction low Mw polymer (/total polymer) - 0.67 | | |

TABLE 6
Formulation 21 - 10 (HCB)/650 (APAP)

| | mg | % |
|---|---|---|
| APAP dense powder | 260.0 | 49.2 |
| Hydrocodone bitartrate | 4.0 | 0.8 |
| Dow PEO WSR 301 LTO (4,000,000) | 32.2 | 6.1 |
| Dow PEO 1105 (900,000) | 64.3 | 12.2 |
| Poloxamer 407 (Lutrol F127) | 33.8 | 6.4 |
| Titanium dioxide (Titanium Dioxide | 10.6 | 2.0 |
| Spectrum USP/EP) | | |
| AO for PEO (BHT) | 0.6 | 0.1 |
| Dicalcium Phosphate | 115.0 | 21.8 |
| Lubricant: Magnesium Stearate | 2.6 | 0.5 |
| Glidant: Colloidal anhydrous silica (Aerosil 200) | 5.3 | 1.0 |
| Total Uncoated Tablet | 528.3 | 100.0 |
| Fraction plasticizer (/total polymer) - 0.35 | | |
| Fraction low Mw polymer (/total polymer) - 0.67 | | |

What is claimed is:

1. A formulation comprising a plurality of discrete mechanically reinforcing particles uniformly dispersed in a pharmaceutically acceptable matrix, wherein said matrix has high fracture toughness and comprises at least one polymer and at least one active agent, at least one abuse-relevant drug or a combination of at least one active agent and at least one abuse-relevant drug.

2. The formulation of claim 1, wherein the polymer has a maximum mechanical energy dissipation at a temperature of about 50° C. or below.

3. The formulation of claim 1, wherein the polymer is semi-crystalline.

4. The formulation of claim 1, wherein the polymer is a semi-crystalline polymer having a maximum mechanical energy dissipation a temperature of about 50° C. or below.

5. The formulation of claim 1, wherein said mechanically reinforcing particles are composed of a filler or a fiber, or of a combination of a filler and a fiber.

6. The formulation of claim 5, wherein the filler is dicalcium phosphate.

7. The method of claim 5, wherein the fiber comprises a cellulosic excipient.

8. The formulation of claim 1, wherein the polymer comprises at least one poly(ethylene oxide).

9. The formulation of claim 8, wherein the poly(ethylene oxide) has a molecular weight of about 100,000 to about 10,000,000 Daltons.

10. The formulation of claim 1, wherein said matrix additionally comprise a plasticizer.

11. The formulation of claim 1, wherein said matrix additionally comprises an anti-oxidant to protective the active agent or abuse-relevant drug.

12. The formulation of claim 10, wherein said plasticizer is a poly(ethylene oxide) having a molecular weight less than about 900,000 Daltons.

13. The formulation of claim 1, wherein the weight ratio of said active agent, abuse-relevant drug or combination of active agent and abuse-relevant drug and the polymer is from about 50:50 to about 0.1 to 99.9.

14. The formulation of claim 1, wherein the abuse-relevant drug is an opioid.

15. The formulation of claim 14, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, levorphanol, lofentanil, meperidine, meptazinol, metazocine, methadone, metopon, morphine, myrophine, nalbulphine, narceine, nicomorphine, norpipanone, opium, oxycodone, oxymorphone, papvretum, paladone, pentazocine, phenadoxone, phenazocine, phenomorphan, phenoperidine, piminodine, propiram, propoxyphene, sufentanil, tapenadol, tilidine, and tramadol, and salts, esters, prodrugs and mixtures thereof.

16. The formulation of claim 14, wherein the opiod is hydrocodone.

17. A method of preparing a formulation, said method comprises the steps of:
a) manufacturing particles which comprise a fracture toughness-imparting polymer and at least one active agent, at least one abuse-relevant drug or a combination of at least one active agent and at least one abuse-relevant drug, and
b) embedding a plurality of said particles in a pharmaceutically acceptable matrix in a unit dosage form.

18. The method of claim 17, wherein said particles comprise a filler or a fiber.

19. The method of claim 18, wherein the filler is dicalcium phosphate.

20. The formulation of claim 10, wherein the plasticizer is poloxamer.

21. The formulation of claim 1, wherein the formulation can be shaped without addition of heat.
